(19) United States
(12) Patent Application Publication
(10) Pub. No.: US 2008/0000001 A1
(43) Pub. Date: Jan. 3, 2008

(54) FACE SHIELD AND METHOD FOR ITS MANUFACTURE

(76) Inventor: Guido Benvenuto, Lasalle (CA)

Correspondence Address: GIFFORD, KRASS, SPRINKLE,ANDERSON & CITKOWSKI, P.C, PO BOX 7021, TROY, MI, 48007-7021, US

(21) Appl. No.: 11/762,847

(22) Filed: Jun. 14, 2007

Related U.S. Application Data
(60) Provisional application No. 60/813,743, filed on Jun. 14, 2006.

(51) Int. Cl. A61F 9/00

(52) U.S. Cl. 2/15

(57) ABSTRACT

Disclosed is a unitary face shield for a sports helmet. The shield includes a first portion which is comprised of a transparent polymeric material and a second portion comprised of a structural polymeric material. The second portion has a plurality of openings defined therethrough, and the first and second portions are fusion bonded together in an edge-to-edge relationship so as to produce a unitary body. The face shield may include still further portions and these may be structural or transparent portions. The transparent portions are fabricated from a high-impact material such as polycarbonate or impact-modified acrylic, and the structural portions are fabricated from high strength structural polymers such as nylon, polypropylene, polycarbonate, ASA, and various combinations. The structural polymer may include reinforcing materials. Also disclosed are methods for manufacturing the shield by composite molding processes.

### CROSS-REFERENCE TO RELATED APPLICATIONS

This application claims priority of U.S. Provisional Patent Application Ser. No. 60/813,743 filed Jun. 14, 2006, entitled “Face Shield and Method for Its Manufacture.”

### FIELD OF THE INVENTION

This invention relates generally to protective devices. More specifically, the invention relates to face shields. Most specifically, the invention relates to face shields fabricated from polymeric materials, and in specific instances relates to face shields used in connection with protective sports headgear such as hockey helmets.

### BACKGROUND OF THE INVENTION

High impact sports such as hockey, football, lacrosse and fencing can present significant risks of harm to a participant's eyes and other portions of the face. As a result, protective gear including faceguards is utilized in many sports. For example, most hockey leagues suggest or mandate that players utilize helmets having attached faceguards.

Heretofore, the practicality and utility of faceguards employed in sporting activities has been limited by the materials and methods available for their fabrication. Prior guards generally relied upon the use of metal mesh grids to protect the wearer's face. These grids are heavy and restrict a user's field of vision thereby compromising sports performance. In some instances, prior art faceguards were fabricated utilizing high strength, transparent polymeric materials; however, face shields made from such materials do not provide adequate ventilation. Consequently, wearers can become overheated, and steaming of the face shield can occur. In some instances, composite face shields have been manufactured utilizing a combination of a transparent portion and a wire mesh portion. However, such face shields still tend to be fairly heavy, and the mechanical junction between the mesh portion and the transparent portion needs to be strong, and this requirement can present design and/or size problems.

As will be explained hereinbelow, the present invention provides a unitary, lightweight, polymeric face shield which combines good visibility with a high degree of ventilation. The shields of the present invention may be configured for various applications as adapted to particular sports. The shields of the present invention are manufactured as unitary bodies comprised of at least two different polymeric materials which are fused together in the course of the fabrication of the shield. The invention will be explained with primary reference to a hockey helmet, although it is to be understood that the principles thereof may be extended to protective devices for other sporting activities such as football, lacrosse and fencing, as well as to safety shields in general such as those used in connection with various industrial, agricultural and logging operations.

### BRIEF DESCRIPTION OF THE INVENTION

Disclosed is a unitary face shield for a sports helmet. The shield includes a first portion comprised of a transparent polymeric material and a second portion comprised of a structural polymeric material. The second portion has a plurality of openings defined therethrough, and the first and second portions are fusion bonded together in an edge-to-edge relationship. The shield may also include at least one more portion fusion bonded in an edge-to-edge relationship with the remainder of the shield. For example, the third portion may be a structural portion fusion bonded, at least in part, to the transparent first portion.

In particular embodiments, the first portion is comprised of a polycarbonate or impact-modified acrylic polymer. The structural polymeric material may comprise, in some instances, nylon, polypropylene, polycarbonate, ASA, and combinations thereof. The transparent first portion may be provided with a hard coating such as a silicon based UV curable coating, or a vapor deposited coating such as carbon based coating or a silicon based coating. The structural polymer may include a reinforcing material therein, and such reinforcing materials may comprise glass fibers, carbon fibers, ceramics, metals, minerals and combinations thereof.

The face shield may be manufactured by a molding process wherein, in a first step, the first portion is injected molded, and wherein, in a second step, the second portion is injected molded in a process wherein it is fusion bonded, in an edge-to-edge relationship with a first portion. Such molding processes may include retractable blade molding, transfer molding, rotational molding or the like.

### BRIEF DESCRIPTION OF THE DRAWINGS

FIG. 1 is a perspective view of a face shield of the present invention as shown being attached to a hockey helmet; and

FIG. 2 is a cross-sectional view of the face shield of FIG. 1 taken along line A-A.

### DETAILED DESCRIPTION OF THE INVENTION

The shields of the present invention are comprised as unitary devices having a first portion fabricated from a transparent polymeric material such as high strength polycarbonate, impact-modified acrylic, and the like. The face shield includes at least a second portion which is comprised of a high strength structural polymeric material such as nylon, polypropylene, polycarbonate, ASA (acrylonitrile styrene acetate) or the like. The foregoing materials may be further reinforced with glass fibers, ceramic fibers, carbon, metals or other such reinforcing materials. This second portion includes a plurality of openings defined therethrough, and is in one embodiment configured as a mesh. During the fabrication of the shield, the second portion is fusion bonded in an edge-to-edge relationship with a first portion so as to create a very strong bond therebetween. Within the context of this disclosure, a fusion bond refers to a joint formed between two bodies of material wherein when the joint is formed when at least one of the materials is molten. The fusion bond provides for a high degree of adherence between the two portions, and may be formed during molding processes, as will be explained in detail hereinbelow, or may be separately formed thereafter.

The face shield may further include yet other portions comprised of structural polymeric materials bonded to the transparent material. For example, a face shield may include a second, ventilating portion having openings formed therethrough, and may also, or alternatively, include attachment portions bonded thereto.

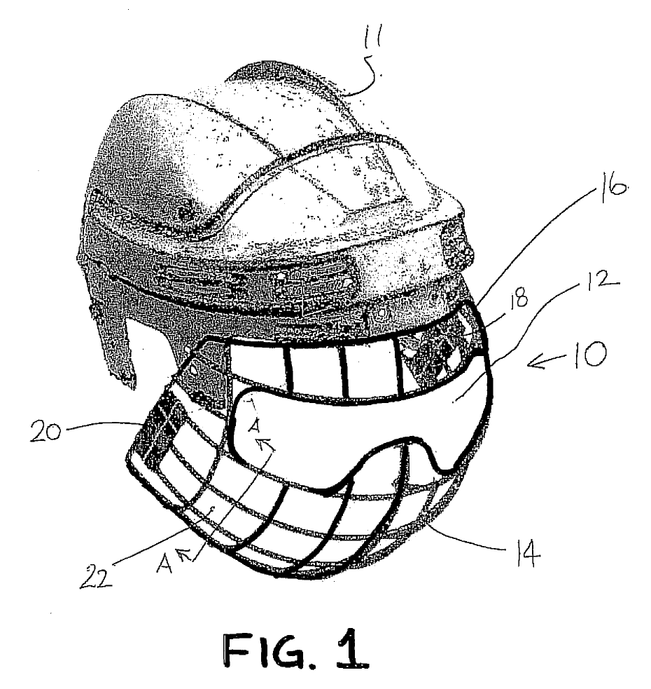

The face shield of the present invention may be implemented in a variety of configurations. FIG. 1 shows one embodiment of face shield 10 structured in accord with the principles of the present invention. As shown, the shield 10 is mounted onto a hockey helmet 11, although it could be mounted onto any other type of protective headgear. The face shield 10 of FIG. 1 includes a first, transparent portion 12 which is formed of a high strength polymer such as polycarbonate or impact-modified acrylic. It is configured and disposed so that when the shield is in use, the first portion will be in the region of the wearer's eyes. The transparent portion 12 may be coated with a high hardness, transparent material so as to increase its resistance to scratching. Such materials may include silicon based coatings such as UV curable coatings, as well as carbon based coatings such as diamond-like carbons deposited by chemical vapor deposition processes.

The face shield 10 of FIG. 1 includes a second portion 14 bonded to the bottom edge of the first portion 12. This second portion 14 is configured to include a plurality of openings defined therein so as to create a meshwork. In the instance where the shield is used as a shield for a hockey helmet, the openings are made sufficiently small so that a hockey puck or hockey stick cannot project very far therethrough. The size and spacing of the openings is optimized to provide maximum protection and ventilation while assuring structural rigidity of the second portion 14. As discussed above, this second portion may be fabricated from a variety of high strength polymers such as filled or unfilled nylon, or polypropylene, fiber-reinforced polycarbonate and the like.

In the FIG. 1 embodiment, a third portion 16 is also bonded to the transparent portion 12. This third portion 16 is also a ventilating portion and, like the second portion 14, includes a plurality of openings defined therethrough. It is generally fabricated from a material which is similar to that used for the second portion. As is further shown in FIG. 1, attachment portions 18 and 20 are also affixed to the face mask 10. As specifically shown, they are joined to the transparent portion 12; although, depending on the configuration of helmet to which the guard is attached, they may be otherwise disposed or configured. The attachment portions 18 and 20 may be fabricated from a high strength polymeric material similar to that used for the second 14 and third 16 portions, or they may be fabricated from other materials, including materials similar to the transparent polymeric material. The attachment portions may include clips, snaps, buckles or the like for joining the shield to a helmet or other headgear. These fasteners may be molded onto the shield, or they may be separate items.

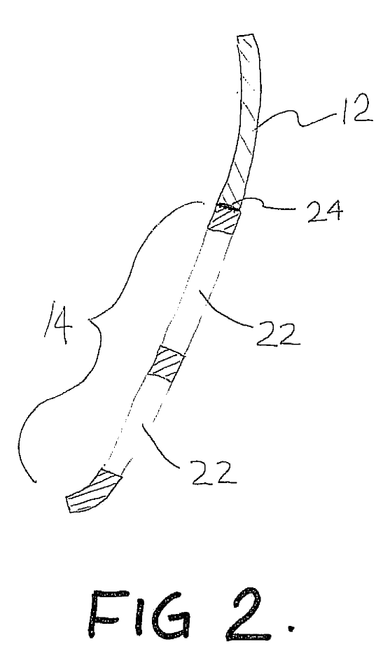

Referring now to FIG. 2, there is shown a cross section of a portion of the faceguard 10 of FIG. 1 taken along line A-A. Specifically, FIG. 2 shows a segment of the transparent portion 12 and a segment of the second portion 14. Visible therebetween is the fusion bond 24. Also visible in FIG. 2 are parts of the openings 22 defined through the second portion 14.

The face shield of the present invention may be manufactured by a number of plastic forming processes which operate to mold articles of two or more different polymers. One method having particular utility in the present invention comprises insert molding. In an insert molding process, as is known in the art, a first member is fabricated in a first molding station in an initial molding process. This member, termed an insert, is then placed in a mold cavity of a second molding station, and a second molding material is injected into the mold cavity so as to fusion bond to, and retain, the insert member.

In a process for the manufacture of a face shield generally similar to that of FIG. 2, the transparent portion 12 may first be formed in an initial forming operation and then inserted into a molding station wherein the second and third portions 14 and 16 are molded thereonto. Obviously, other variations of the process may be employed, as for example the second and/or third portions may comprise the insert, and the transparent portion 12 may be molded thereonto.

Another molding process which may be used to manufacture the composite face shields of the present invention includes processes generally known in the art as a retracting blade injection molding processes. In such processes the molding apparatus includes a mold cavity having a blade or slide member projecting thereinto. This blade or slide member masks off a portion of the mold cavity. In a first step of the process, the blade member is positioned so as to mask the portion of the cavity, and a first material is injected into the unmasked portion of the cavity. Thereafter, the blade or slide member is retracted and a second molding material is then injected into the remainder of the cavity wherein it joins to and forms a fusion bond with the previously injected material. In a process of this type, the transparent portion 12 may be formed in a first step, and the second and third portions formed thereafter. Again, variations of this process will be readily apparent to those of skill in the art.

Yet other processes operative to produce compositely molded polymeric articles, such as rotational molding processes, wherein a series of mold cavities are rotated through one or more molding stations, may also be employed with advantage in the present invention.

While this invention has been described with reference to particularly configured shield and specific molding processes, other modifications and variations of the shield and the process will be readily apparent to those of skill in the art. The foregoing drawings, discussion and description are illustrative of specific embodiments but are not meant to be limitations upon the practice thereof. It is the following claims, including all equivalents, which define the scope of the invention.

1. A unitary face shield for a sports helmet, said shield comprising:
a first portion comprised of a transparent polymeric material; and
a second portion comprised of a structural polymeric material, said second portion being fusion bonded in an edge-to-edge relationship with said first portion, said second portion having a plurality of openings defined therethrough.

2. A face shield as in claim 1, further including a third portion, said third portion comprised of a structural polymeric material and being fusion bonded in an edge-to-edge relationship with said first portion.

3. The face shield of claim 2, wherein said third portion further includes at least one opening defined therethrough.

4. The face shield of claim 1, wherein said first portion is comprised of a polycarbonate or impact-modified acrylic material.

5. The face shield of claim 1, wherein said structural polymeric material is selected from the group consisting of nylon, polypropylene, polycarbonate, ASA, and combinations thereof.

6. The face shield of claim 1, wherein said structural polymeric material includes a reinforcing material disposed therein.

7. The face shield of claim 6, wherein said reinforcing material is selected from the group consisting of glass fibers, carbon fibers, ceramics, metals, minerals, and combinations thereof.

8. The face shield of claim 1, wherein said face shield has been manufactured by a molding process comprising an in-mold transfer process wherein one of said first and second portions is first molded in a first cavity of a mold, then transferred into a second cavity of a mold wherein the other of said first and second portions is molded thereonto.

9. The face shield of claim 1, wherein said face shield is fabricated by an injection molding process.

10. The face shield of claim 9, wherein said injection molding process is carried out in a molding apparatus having a mold cavity and further including a retractable blade which may be disposed in the mold cavity so as to screen a portion of the mold cavity; and wherein one of said first and second portions is molded by a process wherein said blade is projected into said cavity so as to screen said portion of said cavity, and a molten polymeric material is injected into the unscreened portion of the cavity;
and thereafter, the other of said first and second portions is molded by a process wherein the blade is retracted from said cavity, and a second polymeric material is injected into the previously screened portion of the mold cavity so as to form said other of said first and second portion in a fusion bonded relationship with said first or second portion.

11. The face shield of claim 1, wherein said face shield is manufactured by a rotational molding process.

12. The face shield of claim 1, wherein said shield is configured to be attached to a hockey helmet.

13. A method of making a face shield, said method comprising the steps of:
injection molding a first portion of the face shield, said first portion comprised of a transparent polymeric material; and
injection molding a second portion of the face shield, said second portion comprised of a structural polymeric material wherein said second portion is injection molded in an apparatus configured so that the second portion is fusion bonded, in an edge-to-edge relationship, with the first portion.

14. The method of claim 13, wherein said apparatus is a transfer molding apparatus in which an insert comprising the first portion is transferred into a mold cavity wherein molten polymeric material defining said second portion is injected thereinto so as to fusion bond to said first portion.

15. The method of claim 13, wherein said molding process is a retracting blade molding process carried out in a molding apparatus having a mold cavity which defines said first and said second portion, wherein said mold cavity has a retractable blade disposed therein, which retractable blade partitions said mold cavity so as to segregate a first part defining said first portion from a second part defining said second portion and wherein in said first step, molten polymeric material constituting said first portion is injected into the first area of said mold cavity, and thereafter said blade is retracted, and molten polymeric material defining said second portion is injected into the second part of said mold cavity so that the first and second portions are fusion bonded together.
